(19) United States
(12) Patent Application Publication
(10) Pub. No.: US 2021/0261507 A1
(43) Pub. Date: Aug. 26, 2021

(54) Crystalline Form of Fluopyram

(71) Applicant: Bayer Aktiengesellschaft, Leverkusen (DE)

(72) Inventors: Britta Olenik, Bottrop (DE); Birgit Keil, Duesseldorf (DE); Sylvia Dworacek, Solingen (DE); Malcolm Faers, Duesseldorf (DE); Wahed Ahmed Moradi, Monheim (DE); Rolf Gertzmann, Leverkusen (DE)

(21) Appl. No.: 17/263,053

(22) PCT Filed: Jul. 23, 2019
(86) PCT No.: PCT/EP2019/069818

§ 371 Date: Jan. 25, 2021

(30) Foreign Application Priority Data
Jul. 26, 2018 (EP) 18185910.9

(51) Int. Cl. C07D 213/61; A01N 43/40

(52) U.S. Cl. CPC C07D 213/61; C07B 2200/13; A01N 43/40

(57) ABSTRACT

Disclosed are a crystalline form of fluopyram according to formula (1),

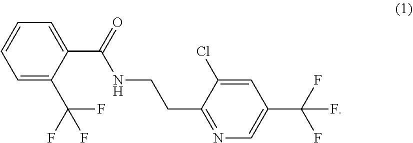 [SMILES: O=C(NCCc1ncc(C(F)(F)F)cc1Cl)c1ccccc1C(F)(F)F]

a process for its preparation agrochemical formulations having the crystalline form, and its use in plant protection applications. Further, an X-ray powder diffractogram at 25° C. and Cu—K□ 1 radiation of the compound displays at least the following reflections, quoted as 2θ value±0.2°: 10.8; 11.5, and 13.5.

### CROSS-REFERENCE TO RELATED APPLICATIONS

This application is the United States national phase of International Application No. PCT/EP2019/069818 filed Jul. 23, 2019, and claims priority to European Patent Application No. 18185910.9 filed Jul. 26, 2018, the disclosures of which are hereby incorporated by reference in their entirety.

### BACKGROUND OF THE DISCLOSURE

### Field of the Disclosure

The present invention relates to a crystalline form of fluopyram according to formula (1),

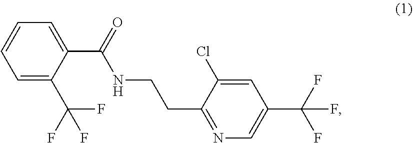 [SMILES: O=C(NCCc1ncc(C(F)(F)F)cc1Cl)c1ccccc1C(F)(F)F]

to a process for its preparation, to agrochemical formulations comprising the crystalline form, and to its use in plant protection applications, especially to its use as a fungicide or nematicide.

### TECHNICAL CONSIDERATIONS

The process known from the prior art yields fluopyram in solid form. Fluopyram has been marketed as a solo product or in combination with other active ingredients as a nematicide or fungicide in different formulations, for example as an Suspension Concentrate (SC), Emulsifiable Concentration (EC) or Flowable Solution (FS) formulations.

The compound according to formula (1) is known under the common name according to ISO as fluopyram (IUPAC name N-{2-[3-chloro-5-(trifluoromethyl)-2-pyridyl]ethyl}-α,α,α-trifluoro-o-tolu amide (CAS No 658066-35-4)). A process for its production is known from WO2004/16088. The process known from the prior art yields fluopyram in a solid form. Fluopyram has been marketed as a solo product or in combination with other active ingredients as a nematicide or fungicide in different formulations, for example as an SC or FS formulation

Active ingredients may present itself in their solid forms both in amorphous or crystalline forms. Amorphous forms lack a long-range order while crystalline forms present a highly-structured microscopic structure having a crystal lattice.

Polymorphism is the ability of a compound to crystallize in different crystalline phases with different arrangements and/or conformations of the molecules in the crystal lattice. Hence, polymorphs are different crystalline forms of the same pure chemical compound. On account of the different arrangement and/or conformation of molecules, amorphous and crystalline forms including polymorphs exhibit different physical, chemical and biological properties. Properties which may be affected include but are not limited to solubility, dissolution rate, stability, optical and mechanical properties, etc. The thermodynamic stability of amorphous and crystalline forms including polymorphs depends on its free energy.

The occurrence of active ingredients in different solid forms like amorphous forms and crystalline forms is of decisive importance for the production in industrial scale as well as for the development of formulations containing the active substance, as unwanted phase change can lead to thickening and potentially solidification of the formulation and/or large crystals, which can lead to blockages in application equipment, e.g. in spray nozzles in agricultural application machinery. The knowledge of the existence of different solid forms like amorphous or crystalline forms and their properties is thus of high relevance. Nevertheless, it is generally not predictable whether a given chemical compound forms amorphous or crystalline forms, in particular polymorph forms at all and if so, which physical and biological properties these forms may have.

In addition pseudopolymophic forms, named hydrates or solvates, can occur. A solvate is a crystalline molecular compound in which molecules of the solvent of crystallisation are incorporated into the host lattice, consisting of unsolvated molecules. A hydrate is a special case of a solvate, when the incorporated solvent is water. The presence of solvent molecules in the crystal lattice influences the intermolecular interactions and confers unique physical properties to each solvate. A solvate thus has its own characteristic values of internal energy, enthalpy, entropy, Gibbs free energy, and thermodynamic activity.

However, the number of amorphous and crystalline forms including polymorph forms for active ingredients is highly variable and there is little scientific insights to what determines the number of amorphous and crystalline forms including polymorph forms. There are also cases known where the formation of polymorphs at least under standard conditions is highly unfavorable so that only one crystalline form of an active ingredient is known. However, it cannot be excluded that under certain conditions also other forms may exist.

Furtheron for active ingredients, in particular fluopyram to give effective biological performance it is important for the fluopyram molecules to be in a solution state to be bioavailable to the target. As an example this can be achieved on a crop by the presence of dew in the morning on plant leaves which can slowly dissolve the active ingredient, in particular fluopyram molecules from the crystalline particles allowing the molecules to distribute over the leaf surface and penetrate inside the leaf. As another example this can be achieved in soil by rain or irrigation water which slowly dissolve the active ingredient molecules, in particular fluopyram molecules from the crystalline particles allowing the molecules to distribute in the soil.

The rate of dissolution of crystals depends on the surface area and can be described by the Noyes-Whitney equation:

$$\frac{d m}{d t} = A \frac{D}{d} ( {C}_{s} - {C}_{t} )$$

where m is the mass of dissolved material, t is time and dm/dt is the dissolution rate, D is the diffusion coefficient of the active ingredient in solution, A is the interfacial surface area of the solid, V is the volume of solution, d is the thickness of the diffusion boundary layer, Cs is the concentration of a saturated solution of the active ingredient at the surface of the crystal and Ct is the concentration of the active ingredient in the bulk medium at time t.

For achieving good bioavailability of an active ingredient it is important for dm/dt to be as high as possible. This can be achieved by increasing the specific surface area A of the crystals, which is the total surface area per unit of mass.

### BRIEF DESCRIPTION OF THE FIGURES

FIG. 1a: X-ray powder diffractogram of a crystalline form of fluopyram

FIG. 1b: FT Raman spectrum of a crystalline form of fluopyram

FIG. 1c: IR spectrum of a crystalline form of fluopyram

FIG. 2: Crystalline form of fluopyram shown in a photograph

FIGS. 3a-3d: Microscopic images of crystal shapes for fluopyram and other different active ingredients illustrating the different aspect ratios; a is fluopyram, b is trifloxystrobin, c is tebuconazole and d is pyrimethanil.

FIGS. 4a-4d: Illustration of different crystal shapes with edge lengths a, b and c.

### DETAILED DESCRIPTION

In a first embodiment, the present invention relates to a crystalline form of fluopyram according to formula (1)

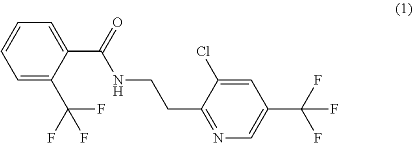 [SMILES: O=C(NCCc1ncc(C(F)(F)F)cc1Cl)c1ccccc1C(F)(F)F]

which represents a thermodynamic stable crystalline form of fluopyram of formula (1) with beneficial physicochemical properties.

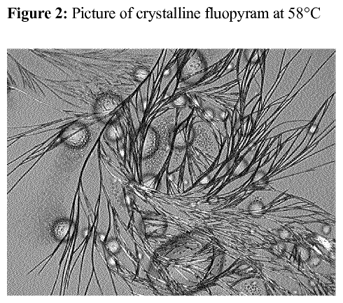

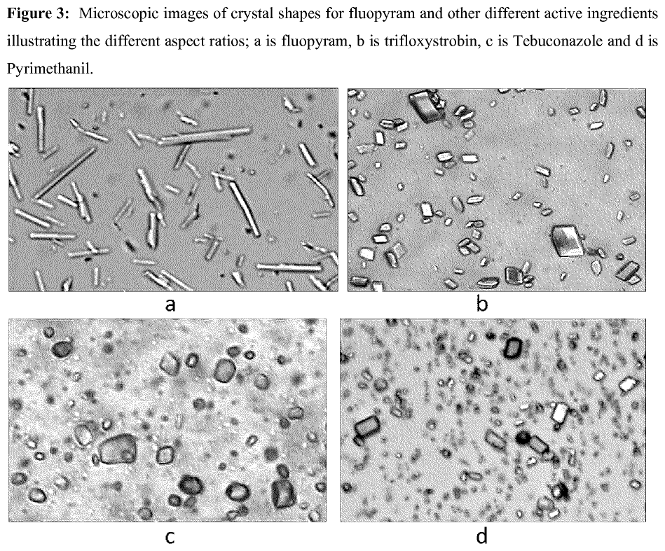

The crystalline form of fluopyram shows a needle-like habit (see FIG. 2). A needle-like habit has a larger surface area compared to other crystalline forms eg the more compact shaped cubic forms as shown in FIG. 3. Therefore fluopyram has enhanced dissolution rates resulting in higher bioavailability after application on plant parts, in particular leaves or in the soil.

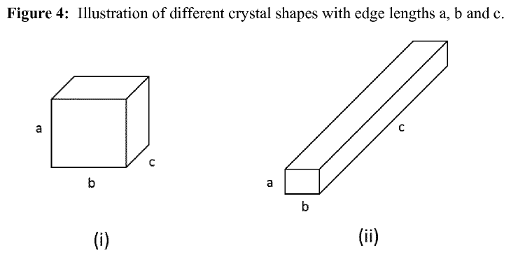

In FIG. 4 (a) illustrates a crystal shape with a low aspect ratio and low surface area and FIG. 4 (b) illustrates a crystal with a high aspect ratio and high surface area for the same mass of material. Illustrations are not accurately drawn to scale.

TABLE 1
Comparison of surface area for crystal shapes with
different edge lengths a, b and c. Volume of
crystal is constant at 1 μm³.

| | | | | Surface | |
|---|---|---|---|---|---|
| Volume | a | b | c | area | Aspect |
| (μm³) | (μm) | (μm) | (μm) | (μm²) | ratio |
| 1 | 1 | 1 | 1 | 1 | 1 |
| 1 | 0.5 | 0.5 | 4 | 7 | 4 |
| 1 | 0.4 | 0.4 | 6.25 | 8.5 | 8 |
| 1 | 0.3 | 0.3 | 11.11 | 10.32 | 15.63 |
| 1 | 0.2 | 0.2 | 25 | 13.51 | 37.04 |
| 1 | 0.1 | 0.1 | 100 | 20.08 | 125 |

The aspect ratio here is defined as the length (c) divided by the width (a or b) since for a 2-dimensional image it is not possible to measure the vertical thickness.

Table 1 demonstrates that for the same crystal volume (i.e. same mass of material), crystals with high aspect ratios have higher surface areas.

In order to produce agrochemical formulations, in particular in SC formulations the crystalline form of fluopyram is milled to a particle size of 0.1 to 50 microns, preferably 0.5 to 25 microns and most preferably 1 to 15 microns or 1 to 10 microns.

The crystalline form of fluopyram can be characterized by X-ray powder diffractometry on the basis of the respective diffraction diagrams, which are recorded at 25° C. and with Cu—Kα 1 radiation (1.5406 Å). The crystalline form displays at least three, often at least five, in particular at least seven, more particularly at least ten, and especially all of the reflections quoted in the following as values:

TABLE 2
X-ray reflections of the crystalline
form of fluopyram
Reflections [2θ values]
x ± 0.2°

5.4
8.2
9.8
10.8
11.5
13.5
16.4
16.7
17.2
18.2
18.6
19.2
19.8
20.0
20.5
21.5
22.0
22.5
22.6
22.9
23.2
24.6
25.0
25.7
26.5
26.9
27.5
28.0
28.6
29.4
30.8
31.7
33.0
37.0

The crystalline form is further characterized by the X-ray powder diffractogram depicted in FIG. [1a].

The crystalline form of fluopyram can be characterized by Raman spectroscopy on the basis of the respective spectrum, which are recorded at 25° C. and with a laser wavelength of 1064 nm and a resolution of 2 cm⁻¹. The crystalline form of fluopyram displays at least 3, often at least five, in particular at least seven, and especially all of the bands quoted in the following as peak maxima:

TABLE 3
Raman bands of the crystalline
form of fluopyram
Raman band [peak maxima in cm⁻¹]

3268
3098
3074
3041
2946
2904
1642
1606
1588
1555
1451
1427
1376
1331
1314
1286
1272
1223
1202
1172
1134
1096
1072
1036
882
195
169
717
683
647
599
560
461
432
393
357
288
257
232
184
153
124

The crystalline form of fluopyram can be characterized by infrared spectroscopy on the basis of the respective spectrum, which are recorded at 25° C. using an universal diamond ATR device and a resolution of 4 cm⁻¹. The crystalline form of fluopyram displays at least three, often at least five, in particular at least seven, and especially all of the bands quoted in the following as peak maxima:

TABLE 4
IR bands of the crystalline form
of fluopyram
IR band [peak maxima in cm⁻¹]

3264
3086
1639
1606
1588
1551
1493
1466
1449
1399
1377
1332
1314
1284
1273
1224
1200
1176
1152
1126
1111
1094
1071
1056
1035
959
919
883
869
767
750
706
682
646
641
599
589
559

In one embodiment a process (A) for the production of the crystalline form is described, comprising the following steps:

A1) 2,3-dichloro-5-trifluoromethyl-pyridine and dimethyl-malonate are added to a solution of potassium hydroxide and dimethylacetamide resulting in the formation of Dimethyl [3-chloro-5-(trifluoromethyl)pyridin-2-yl]malonate.
A2) Dimethyl [3-chloro-5-(trifluoromethyl)pyridin-2-yl]malonate and {[2-(trifluoromethyl)benzoyl]amino}methyl acetate are reacted in the presence of acetic acid into Dimethyl [3-chloro-5-(trifluoromethyl)pyridin-2-yl]({[2-(trifluoro-methyl)benzoyl]amino}methyl)malonate.
A3) Dimethyl [3-chloro-5-(trifluoromethyl)pyridin-2-yl]({[2-(trifluoro-methyl)benzoyl]amino}methyl)malonate is saponified in the presence of caustic soda.
A4) In the presence of methanol the reaction mixture of step A3) is acidified by adding hydrochloric acid inducing the crystallization and precipitation of fluopyram which can then be separated by filtration.

In another embodiment, a process (B) for the production of the crystalline form is described, comprising the following steps:
B1) heating a sample of solid fluopyram to a temperature between 115 and 120° C.; and
B2) cooling the melted fluopyram obtained in step b) with a cooling rate which is preferably less than 10 K/min to a temperature of less than 100° C.

The chemical preparation of fluopyram according to formula (1) is known from WO2004/16088, WO2018/114484 and WO2015/071230. The compound of formula (1) as used in step B1) or A4) can thus be prepared according to WO2004/16088, WO2018/114484 and WO2015/071230, to which full reference is made hereby.

Suitable solvents or solvent mixtures which can be used to dilute and/or suspend the compound of formula (1) in step A4) and from which the compound of formula (1) is obtained in crystalline form in step A4), are petroleum ether, hexane, heptane, cyclo-hexane, methyl-cyclohexane, benzene, toluene, xylene, decalin, chloro-benzene, dichloro-benzene, trifluoromethyl benzene, dichloromethane, chloroform, carbon tetra-chloride, di-chlorethane, tri-chlor-ethane, diethyl ether, diisopropyl ether, methyl tert-butyl-ether, methyl tert-amyl-ether, cyclopentyl-methyl-ether, dioxane, tetrahydrofuran, methyl tetrahydrofuran, 1,2-di-methoxyethane, 1,2-di-ethoxy-ethane, anisole, N,N-dimethyl-formamide, N,N-dimethyl-acetamide, N-methyl-formanilide, acetonitrile, butyronitrile, methanol, ethanol, isopropanol, 1-propanol, 2-methoxy ethanol, tert. butanol, 1-butanol, 2-butanol, cyclohexanol, ethandiole, ethylene glycol, N-methyl-pyrrolidone, hexamethyl-phosphoric-triamide or 1,3-dimethyl-2-2-imidazolinone or N,N-dimethyl acetamide (DMAC).

In step A2) the solution or slurry is usually heated to a temperature of at least 115° C., preferably to a temperature of at least 120° C., and most preferably to a temperature of 125° C. In a preferred embodiment each solvent or solvent mixture is heated to its boiling temperature.

In step B2) the solution or slurry is cooled to a temperature of less than 105° C., preferably less than 100° C., and preferably to a temperature of 90° C.

The isolation of the crystalline form from the mother liquid is effected by common techniques known in the art, for example by filtration, centrifugation or by decanting. The isolated crystalline form can optionally be washed with any solvent, preferably with the solvent or solvent mixture used for crystallization, with water or with a mixture of the solvent or solvent mixture and water. The washing step can optionally be repeated, whereby washing with water often is the last washing step. The washing is typically performed at temperatures below 30° C., often below 25° C. and in particular below 20° C., optionally at 0° C. In a further, optionally step, the crystals of crystalline form can be dried and then supplied for further processing.

By means of the crystallization, the crystalline form of fluopyram is obtained with at least 85%, in particular 90%, and most preferably at least ≥95% from process A).

The content of the crystalline form of fluopyram is analyzed by Raman spectroscopy. Based on calculated electronically mixed Raman spectra (mixed by a software calculator in 5% steps) a calibration curve, using a PLS regression, is generated. fluopyram

In a third embodiment, the present invention is directed to a plant protection agent in the form of customary formulations containing the crystalline form of fluopyram.

The plant protection agent may additionally comprise one or more further active substance(s) selected from the group consisting of herbicides, insecticides, acaricides, fungicides, safeners and/or plant growth regulator. The plant protection agent may further comprise adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulfate or diammonium hydrogenphosphate and/or retention promoters, for example dioctyl sulfosuccinate or hydroxypropylguar polymers and/or humectants, for example glycerol and/or fertilizers, for example ammonium-, potassium- or phosphorus-containing fertilizers.

Customary formulations are, for example, suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers 173, prepared by the FAO/WHO Joint Meeting on Pesticide Specifications, 2004, ISBN: 9251048576.

Preference is given to formulations or use forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration. These formulations are prepared in a known way, for example by mixing the compounds of the formula (I) with auxiliaries such as, for example, extenders, solvents and/or solid carriers and/or other auxiliaries such as, for example, surfactants. The formulations are produced either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and water.

In principle, it is possible to use all suitable solvents. Examples of suitable solvents are aromatic hydrocarbons, such as xylene, toluene or alkylnaphthalenes, chlorinated aromatic or aliphatic hydrocarbons, such as chlorobenzene, chloroethylene or methylene chloride, aliphatic hydrocarbons, such as cyclohexane, paraffins, mineral oil fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulfoxide, and also water.

In principle, it is possible to use all suitable carriers. Useful carriers especially include: for example ammonium salts and ground natural minerals such as kaolins, clays, talc, chalk, quartz, attapulgite, montmorillonite or diatomaceous earth, and ground synthetic minerals such as finely divided silica, alumina and natural or synthetic silicates, resins, waxes and/or solid fertilizers. It is likewise possible to use mixtures of such carriers. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, corn cobs and tobacco stalks. It is also possible to use liquefied gaseous extenders or solvents. Especially suitable are those extenders or carriers which are gaseous at standard temperature and under atmospheric pressure, for example aerosol propellants such as halogenated hydrocarbons, and also butane, propane, nitrogen and carbon dioxide. Examples of emulsifiers and/or foam formers, dispersants or wetting agents having ionic or nonionic properties or mixtures of these surfactants are salts of polyacrylic acid, salts of lignosulfonic acid, salts of phenolsulfonic acid or naphthalenesulfonic acid, polycondensates of ethylene oxide with fatty alcohols or with fatty acids or with fatty amines, with substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolyzates, lignosulfite waste liquors and methylcellulose. The presence of a surfactant is advantageous if one of the compounds of the formula (I) and/or one of the inert carriers is insoluble in water and when the application takes place in water.

Further auxiliaries which may be present in the formulations and the use forms derived therefrom nutrients and trace nutrients such as salts of iron, manganese, boron, copper, cobalt, molybdenum and zinc. Additional components which may be present are stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further auxiliaries may be mineral and vegetable oils.

It is possible if appropriate for still further auxiliaries to be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce dynamic surface tension, for example dioctyl sulfosuccinate, or increase viscoelasticity, for example hydroxypropylguar polymers.

The crystalline form has improved formulation properties since after application onto the plant, plant parts or soil fluopyram exists as crystalline particles with a needle-like habit. These forms show an increased rate of dissolution from increased surface area with the needle crystal form compared to other forms, eg cubic forms. In another embodiment, the present invention is therefore directed to the use of the crystalline form of the compound of formula (1) for the production of a formulation providing after application the active ingredient, in particular fluopyram with an increased crystal surface area and enhanced rate of dissolution. This may be illustrated using the Noyes-Whitney equation.

All plants and plant parts can be treated. By plants is meant all plants and plant populations such as desirable and undesirable wild plants, cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed mutagenesis, molecular or genetic markers or by bioengineering and genetic engineering methods.

By plant parts is meant all above ground and below ground parts and organs of plants such as shoot, leaf, blossom and root, whereby for example leaves, needles, stems, branches, blossoms, fruiting bodies, fruits and seed as well as roots, corms and rhizomes are listed. Crops and vegetative and generative propagating material, for example cuttings, corms, rhizomes, runners, slips and seeds also belong to plant parts. Preferred plant parts are leaves, roots and seeds.

### Working Examples

### Methods

All data which is part of the present application has been prepared according to the methods described below unless otherwise indicated. The samples used for measurement were directly used and did not undergo any further sample preparation.

### XRPD

X-Ray diffraction patterns were recorded at room temperature using XRD diffractometers X'Pert PRO (PANalytical) and STOE STADI-P (radiation Cu K alpha 1, wavelength 1.5406 Å). All X-Ray reflections are quoted as °2θ (theta) values (peak maxima) with a resolution of ±0.2°.

### Raman

Raman spectra were recorded at room temperature using FT-Raman-spectrophotometers (model RFS 100 and MultiRam) from Bruker. Resolution was 2 cm⁻¹. Measurements were performed in glass vials or aluminium discs.

### IR

IR-ATR-spectra were recorded at room temperature using a FT-IR-spectrophotometer one with universal diamond ATR device from Perkin-Elmer. Resolution was 4 cm⁻¹.

### I Crystalline Form of Fluopyram

### I.1 Preparation of the Crystalline Form of Fluopyram

### Step 1: Dimethyl [3-chloro-5-(trifluoromethyl)pyridin-2-yl]malonate (Salt Free Compound) [=Py-Malonester]

A suspension of 71.8 g potassium hydroxide [KOH] in N,N-dimethyl acetamide (DMAC) was heated to app. 60° C. 180.1 g of a pre-mixed solution of dimethyl-malonate [DMM] and 2,3-dichloro-5-trifluoromethyl-pyridine [PyCl], (70.9 g DMM and 109.2 g PyCl) was added over several hours. At the end of the addition of [DMM/PyCl] a yellow solid precipitates out of the solution in particular after cooling down the suspension to room temperature.

### Step 2: Dimethyl [3-chloro-5-(trifluoromethyl)pyridin-2-yl]({[2-(trifluoro-methyl)benzoyl]amino}methyl)malonate [=Py-Diester]

12 g acetic acid is added at 60° C. to the suspension of [Py-Malonester] from step 1. Subsequently, a solution of 254.6 g {[2-(trifluoromethyl)benzoyl]amino}methyl acetate [TFMB-acetate] is added at 60° C. The suspension is stirred for several hours at 80° C. The solvent DMAC is removed by distillation under reduction of the temperature. The residue, consisting mainly of [Py-Diester] and inorganic salts, is dissolved at 50° C. in water and methyl tert-butyl ether (MTBE). After phase separation the MTBE phase is transferred to the next step without further treatment.

### Step 3: 2-[3-chloro-5-(trifluoromethyl)pyridin-2-yl]-3-{[2-(trifluoromethyl)-benzoyl]amino}propanoic acid (Salt Free Compound) [Py-Na-Salt]

Water is added to the MTBE solution of the [Py-Diester]-step. Afterwards, caustic soda (32 wt. %) is added to the MTBE-water mixture in 2 hours keeping the temperature at 35° C. followed by additional stirring at 35° C. After complete saponification the aqueous phase is separated. Additional water is added to the MTBE solution at 35° C. and heated to ca. 60° C. MTBE is removed by distillation at 60° C. under vacuum.

### Step 4: N-{2-[3-chloro-5-(trifluoromethyl)pyridin-2-yl]ethyl}-2-(trifluoro-methyl)benzamide [fluopyram]

Methanol is added to the reaction mixture of step 3 at ca. 52° C. followed by the addition of hydrochloric acid (20 wt %) at 52° C. until the mixture reaches a pH between 2 and 3. fluopyram starts to form the crystalline form around pH 5. Finally the product is separated by filtration and washed with a mixture of MeOH/water. The wet cake is dried.

174.5 g fluopyram (98.4 wt. %) with 86.6% chemical yield was obtained.

The obtained crystals of the crystalline form of fluopyram were isolated and analyzed by X-ray powder diffraction (XRPD), Raman and IR.

### II Properties of Crystalline Form of Fluopyram of Formula (1)

The crystallization behavior of fluopyram was analyzed using cyclic Differential Scanning Calometry in a temperature range from 25° C. up to 130° C. with 10 k/min range of heating.

fluopyram has a melting point of 118 to 120° C. and shows needle-like habits upon recrystallization. Recrystallization starts already below 115° C. The speed of recrystallization is very fast below 105° C., in particular below 103° C. Additional heating and cooling periods do not change the melting/crystallization behavior of fluopyram. Upon repeated heating and cooling no differences in melting point (118° C.; 74 J/g) were detected. The needle-like structures are shown in FIG. 2.

### Preparation of Formulations Containing Needle Shaped Crystals of Fluopyram.

In an example 500 g/L of fluopyram crystalline active ingredient was added to a mixture of 65 g/L wetters and dispersants, 80 g/L propylene glycol, 2 g/L silicone antifoam in 432 g/L with high shear mixing (Ultra-Turrax®) to reduce the particle size D(v,0.9) to approximately 50 microns, then passed through a bead mill (Eiger® 250 Mini Motormill) to achieve a particles size D(v,0.9) typically 1 to 15 microns. Then a gel composed of 2.4 g/L xanthan and 1 g/L biocide in 117 g/L water was added with low shear mixing (stirrer). The resulting suspension concentrate contained fluopyram crystals which after ageing for 1 month at 40 to 45° C. showed a needle-like appearance, the needle-like habit.

In an example an SC of fluopyram may also be used to create whole granule (WG) formulations by for example extrusion or spray drying or GR formulations by coating a granular carrier substrate.

In an example an EC formulation was prepared by dissolving 1 to 500 g/L of fluopyram in a mixture comprising a certain amount of emulsifiers and a certain amount of solvent. The resulting EC formulation produced needle shaped crystals after dilution at 1% in water and evaporation on the leaf surface.

In an example the aspect ratio for crystals of fluopyram and other different active ingredients was measured from microscopic images taken from suspension concentrate formulations after crystal growth had occurred, diluted to approximately 1% in water. For the purpose of this invention the length is defined as the longest dimension and the width as the shortest dimension of a 2-dimensional image obtained in a transmission optical microscope. The aspect ratio was determine by the dividing the length by the width.

In table 5 the comparison of the surface area of different crystalline forms is shown.

TABLE 5.1
Aspect ratios of fluopyram crystals
present in suspension concentrate formulations
(numbers are rounded).

| Active | Length | Width | Aspect |
|---|---|---|---|
| ingredient | (μm) | (μm) | ratio |
| fluopyram | 24.3 | 2.0 | 12.15 |
| | 16.4 | 1.4 | 11.71 |
| | 14.6 | 1.1 | 13.27 |
| | 14.2 | 1.9 | 7.47 |
| | 30.7 | 1.6 | 19.19 |
| | 11.5 | 1.0 | 11.50 |
| | 11.1 | 0.9 | 12.33 |
| | 10.0 | 0.9 | 11.11 |
| | 19.6 | 1.8 | 10.89 |
| | 15.8 | 1.5 | 10.53 |
| mean | | | 12.02 |

TABLE 5.2
Aspect ratios of trifloxystrobin crystals present
in suspension concentrate formulations (numbers
are rounded).

| Active | Length | Width | Aspect |
|---|---|---|---|
| ingredient | (μm) | (μm) | ratio |
| trifloxystrobin | 10.0 | 8.4 | 1.19 |
| | 4.1 | 2.9 | 1.41 |
| | 6.4 | 4.9 | 1.31 |
| | 5.8 | 5.6 | 1.04 |
| | 5.0 | 4.6 | 1.09 |
| | 5.0 | 3.6 | 1.39 |
| | 4.1 | 3.9 | 1.05 |
| | 10.3 | 6.4 | 1.61 |
| | 5.3 | 4.8 | 1.10 |
| | 6.4 | 4.9 | 1.31 |
| Mean | | | 1.25 |

TABLE 5.3
Aspect ratios of tebuconazole crystals present
in suspension concentrate formulations
(numbers are rounded).

| Active | Length | Width | Aspect |
|---|---|---|---|
| ingredient | (μm) | (μm) | ratio |
| tebuconazole | 7.2 | 6.7 | 1.07 |
| | 5.6 | 3.9 | 1.44 |
| | 4.0 | 4.0 | 1.00 |
| | 9.5 | 7.9 | 1.20 |
| | 8.6 | 6.6 | 1.30 |
| | 5.1 | 5.0 | 1.02 |
| | 5.4 | 5.3 | 1.02 |
| | 10.1 | 8.3 | 1.22 |
| | 6.5 | 4.6 | 1.41 |
| | 7.9 | 9.5 | 0.83 |
| mean | | | 1.15 |

TABLE 5.4
Aspect ratios of pyrimethanil crystals present
in suspension concentrate formulations
(numbers are rounded).

| Active | Length | Width | Aspect |
|---|---|---|---|
| ingredient | (μm) | (μm) | ratio |
| pyrimethanil | 5.7 | 5.0 | 1.14 |
| | 6.6 | 4.6 | 1.43 |
| | 5.2 | 3.4 | 1.53 |
| | 8.9 | 6.1 | 1.46 |
| | 5.2 | 3.3 | 1.58 |
| | 8.0 | 7.1 | 1.13 |
| | 4.5 | 2.8 | 1.61 |
| | 4.9 | 4.8 | 1.02 |
| | 6.3 | 3.2 | 1.97 |
| | 6.5 | 4.1 | 1.59 |
| Mean | | | 1.44 |

FIGS. 3a-3d illustrate the needle-like habit of fluopyram in contrast to the cubic shaped forms of crystals of trifloxystrobin, tebuconazole and pyrimethanil.

TABLE 6
Aspect ratios of different active ingredient
crystals present in suspension concentrate
formulations from tables 5.1 to 5.4

| Active ingredient | Aspect ratio |
|---|---|
| fluopyram | 12.02 |
| trifloxystrobin | 1.25 |
| tebuconazole | 1.15 |
| pyrimethanil | 1.44 |

The results in Table 6 demonstrate that fluopyram crystals show a substantially higher aspect ratio than many other active ingredients and therefore have higher surface areas than many other active ingredient crystals for the same mass of material per crystal.

### III XRPD Data of the Crystalline Form of Fluopyram

X-Ray diffraction patterns were recorded at room temperature using XRD diffractometers X'Pert PRO (PANalytical) and STOE STADI-P (radiation Cu K alpha 1, wavelength 1.5406 Å). All X-Ray reflections are quoted as °2θ (theta) values (peak maxima) with a resolution of ±0.2°.

### Measurement Parameters:

Powder pattern were recorded at room temperature using powder diffractometer (model X'PERT PRO) from PANalytical. Measurements were performed in transmission mode between two acetate foils under following conditions:

Generator: 40 kV/40 mA

### Radiation: CuKα (1.54 Å)

Scan range: 2 40°2θ
Scan step: 0.013°2θ
Scan time: 25 sec/step

TABLE 1
Reflections of the crystalline form of fluopyram
Reflections
(Peak maxima)
[2 Theta]
Crystalline form

5.4
8.2
9.8
10.8
11.5
13.5
16.4
16.7
17.2
18.2
18.6
19.2
19.8
20.0
20.5
21.5
22.0
22.5
22.6
22.9
23.2
24.6
25.0
25.7
26.5
26.9
27.5
28.0
28.6
29.4
30.8
31.7
33.0
37.0

### Characteristic Reflections:

The following reflections are considered characteristic for the crystalline form of fluopyram: preferably 10.8; 11.5 and 13.5;

more preferably at least the following reflections: 10.8; 11.5; 13.5; 16.4 and 16.7;
even more preferably at least the following reflections: 10.8; 11.5; 13.5; 16.4; 16.7; 20.0 and 22.0;
most preferably at least the following reflections: 10.8; 11.5; 13.5; 16.4; 16.7; 20.0; 22.0; 22.5; 24.6 and 25.0, each quoted as °2θ value±0.2°.

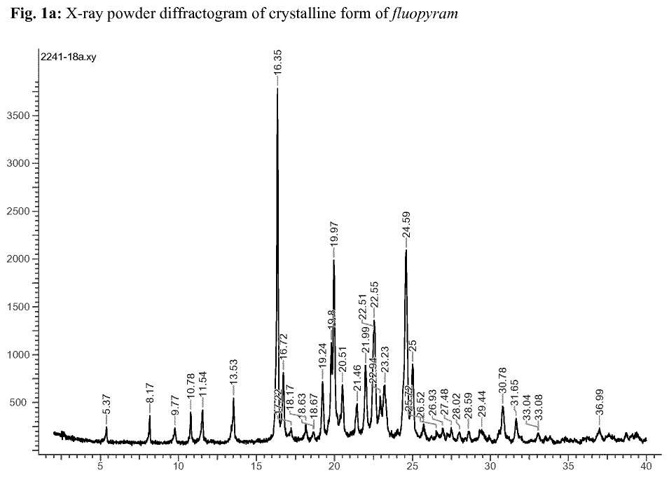

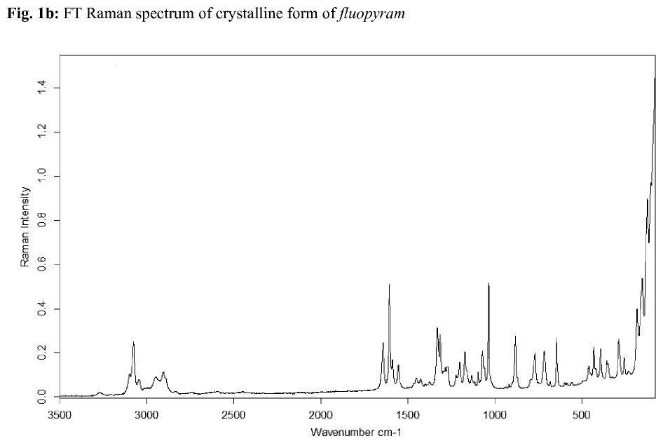

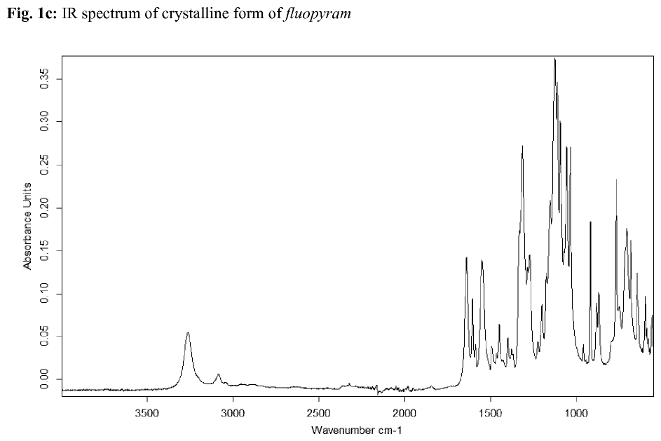

FIG. 1a shows the X-ray powder diffractogram of the crystalline form of fluopyram.

### III Raman Data of the Crystalline Form of Fluopyram

Raman spectra were recorded at room temperature using FT-Raman-spectrophotometers (model RFS 100 and MultiRam) from Bruker. Resolution was 2 cm¹. Measurements were performed in glass vials or aluminium discs. There was no sample preparation.

The crystalline form of fluopyram can be characterized by Raman spectroscopy on the basis of the respective spectrum, which are recorded at 25° C. and with a laser wavelength of 1064 nm and a resolution of 2 cm⁻¹. The crystalline form of fluopyram displays at least three, often at least five, in particular at least seven, and especially all of the bands quoted in the following as peak maxima:

TABLE 3
Raman bands of the crystalline
form of fluopyram
Raman band [peak maxima in cm⁻¹]

3268
3098
3074
3041
2946
2904
1642
1606
1588
1555
1451
1427
1376
1331
1314
1286
1272
1223
1202
1172
1134
1096
1072
1036
882
195
169
717
683
647
599
560
461
432
393
357
288
257
232
184
153
124

### Characteristic Bands:

The following bands are considered characteristic for the crystalline form of fluopyram:

preferably 3074, 1642 and 1606;
more preferably at least the following bands: 3074, 1642, 1606, 1331 and 1314;
even more preferably at least the following bands: 3074, 1642, 1606, 1331, 1314, 1036 and 882; most preferably at least the following bands: 3074, 1642, 1606, 1331, 1314, 1036, 882, 769, 717 and 124, each quoted in cm⁻¹ value±2 cm⁻¹.
FIG. 1b shows the Raman spectra of the crystalline form of fluopyram.

### III Infrared Data of the Crystalline Form of Fluopyram

IR-ATR-spectra were recorded at room temperature using a FT-IR-spectrophotometer one with universal diamond ATR device from Perkin-Elmer. Resolution was 2 cm⁻¹. There was no sample preparation.

TABLE 4
IR bands of the crystalline form
of fluopyram
IR band [peak maxima in cm⁻¹]

3264
3086
1639
1606
1588
1551
1493
1466
1449
1399
1377
1332
1314
1284
1273
1224
1200
1176
1152
1126
1111
1094
1071
1056
1035
959
919
883
869
767
750
706
682
646
641
599
589
559

### Characteristic Bands:

The following bands are considered characteristic for the crystalline form of fluopyram:

### Preferably 3264, 1639 and 1551;

more preferably at least the following bands: 3264, 1639, 1551, 1314 and 1126;
even more preferably at least the following bands: 3264, 1639, 1551, 1314, 1126, 1111 and 1094;
most preferably at least the following bands: 3264, 1639, 1551, 1314, 1126, 1111, 1094, 1056, 1035 and 767, each quoted in cm⁻¹ value±2 cm⁻¹.

FIG. 1c shows the Infrared spectra of the crystalline form of fluopyram.

1. A crystalline form of a fluopyram compound comprising a crystalline form of a compound of formula (1), 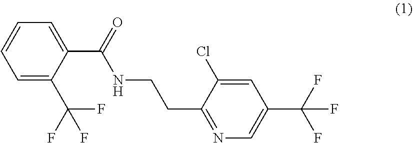 [SMILES: O=C(NCCc1ncc(C(F)(F)F)cc1Cl)c1ccccc1C(F)(F)F]
wherein an X-ray powder diffractogram at 25° C. and Cu—K□ 1 radiation of the compound displays at least the following reflections, quoted as 2θ value±0.2°: 10.8; 11.5; and 13.5.

2. The crystalline form of the fluopyram compound of claim 1, wherein the X-ray powder diffractogram at 25° C. and with Cu—K□ 1 radiation of the compound displays at least the following reflections, quoted as 2θ value±0.2°: 10.8; 11.5; 13.5; 16.4; and 16.7.

3. The crystalline form of the fluopyram compound of claim 1, wherein the X-ray powder diffractogram at 25° C. and with Cu—K□ 1 radiation of the compound displays at least the following reflections, quoted as 2θ value±0.2°: 10.8; 11.5; 13.5; 16.4; 16.7; 20.0; and 22.0.

4. The crystalline form of the fluopyram compound of claim 1, wherein a Raman spectrum of the compound displays at least the following bands (peak maximum in cm⁻¹): 3074, 1642, and 1606.

5. The crystalline form of the fluopyram compound of claim 1, wherein a Raman spectrum of the compound displays at least the following bands (peak maximum in cm-1): 3074, 1642, 1606, 1331, and 1314.

6. The crystalline form of the fluopyram compound of claim 1, wherein a Raman spectrum of the compound displays at least the following bands (peak maximum in cm-1): 3074, 1642, 1606, 1331, 1314, 1036, and 882.

7. The crystalline form of the compound of claim 1, wherein an infrared spectrum of the compound displays at least the following bands (peak maximum in cm-1): 3264, 1639, and 1551.

8. The crystalline form of the fluopyram compound of claim 1, wherein an infrared spectrum of the compound displays at least the following bands (peak maximum in cm-1): 3264, 1639, 1551, 1314, and 1126.

9. The crystalline form of the fluopyram compound of claim 1, wherein an infrared spectrum of the compound displays at least the following bands (peak maximum in cm-1): 3264, 1639, 1551, 1314, 1126, 1111, and 1094.

10. A process for the production of a crystalline form of a fluopyram compound, the process comprising:
B1) heating a sample of a solid compound of formula (1) of claim 1 to a temperature between 115° C. and 120° C.; and
B2) cooling a melted compound of formula (1) of claim 1 obtained in step B1) with a cooling speed which is less than 10 K/min to a temperature of less than 105° C.

11. The process according to claim 10, wherein the heating temperature of step B1) is at least 115° C.

12. The process according to claim 10, wherein a solution or slurry of step B2) is cooled to a temperature of less than 105° C.

13. A plant protection agent containing the crystalline form of the compound of formula (1) of claim 1.

14. The plant protection agent according to claim 13, further comprising one or more agriculturally acceptable additive customary for the formulation of plant protection agents.

15. The plant protection agent according to claim 13, further comprising one or more additional active substances selected from the group consisting of herbicides, insecticides, acaricides, fungicides, safeners, plant growth regulators, and combinations thereof.
